(19) United States
(12) Patent Application Publication
(10) Pub. No.: US 2021/0085582 A1
(43) Pub. Date: Mar. 25, 2021

(54) SYNERGISTIC ANTIOXIDANT ACID COMPOUND ASSOCIATIONS

(71) Applicant: L'OREAL, Paris (FR)

(72) Inventors: Stephen Matthew LYNCH, Westfield, NJ (US); Ben GOODYEAR, Clark, NJ (US); Patricia BRIEVA, Manalapan, NJ (US)

(73) Assignee: L'OREAL, Paris (FR)

(21) Appl. No.: 16/583,247

(22) Filed: Sep. 25, 2019

(51) Int. Cl. A61K 8/368; A61K 8/67; A61K 8/365; A61K 8/06

(52) U.S. Cl. CPC A61K 8/368; A61K 8/676; A61K 2800/522; A61K 8/064; A61K 8/068; A61K 8/365

(57) ABSTRACT

A composition that includes a novel association of antioxidant acids demonstrates an unexpected synergistic antioxidant effect that is relevant for the topical treatment of signs of aging and reducing cutaneous lipid peroxidation. The composition includes (a) gallic acid, (b) ferulic acid, (c) ascorbic acid (Vitamin C), and (d) salicylic acid, all present in the composition in amounts sufficient to produce the synergistic antioxidant activity. In some embodiments, gallic acid is present in an amount that is in the range from about 1% to about 10%, and in some examples, in an amount that is at least about 5%.

### FIELD OF THE INVENTION

The invention relates to a novel association of antioxidant acids that demonstrates an unexpected synergistic antioxidant effect that is relevant for the topical treatment of signs of aging and reducing cutaneous lipid peroxidation. The novel association may be particularly beneficial for treating oily and/or acne prone skin

### BACKGROUND OF THE INVENTION

The formation of free radicals is widely considered to play a significant role in the mechanisms of skin aging. Free radicals are highly reactive molecules with unpaired electrons that can directly damage various cellular membranes, lipids, proteins, RNA and DNA. The damaging effects of these reactive oxygen species are induced inside the tissue and cells during normal metabolism and externally through various oxidative stresses. UV exposure and environmental pollution can accelerate skin aging by producing free radicals in skin. Antioxidants protect cells from the damage of oxidative stress by scavenging free radicals and inhibiting oxidation reactions. The topical application of antioxidants is broadly used in skin care products to prevent skin aging. It has been previously shown in the cosmetic related fields that polyphenols act synergistically with other antioxidants such as Vitamin E and carotenoids.

Despite these developments, there is continued interest in identifying antioxidant compound associations that can more meaningfully enhance the efficacy of topical treatment compositions to protect and rejuvenate skin and address the signs of aging including reducing cutaneous lipid peroxidation.

### BRIEF SUMMARY OF THE INVENTION

The invention provides a composition comprising (a) gallic acid, (b) ferulic acid, (c) ascorbic acid (Vitamin C), and (d) salicylic acid, where (a)-(d) are present in the composition in amounts sufficient to produce synergistic antioxidant activity. In some embodiments, gallic acid is present in an amount that is in the range from about 1% to about 10%. In some specific embodiments, gallic acid is present in an amount that is at least about 5%.

In some embodiments, gallic acid is present in the composition in a range from about 1% to about 10%, by weight, based on the weight of the composition. In some embodiments, the amount of gallic acid present in the composition is in an amount that is at least about 5% to about 10%, by weight, based on the weight of the composition. In some embodiments, the amount of gallic acid present in the composition is in an amount that is at least about 5%, by weight, based on the weight of the composition.

In some embodiments, ferulic acid is present in the composition in a range from about 0.1% to about 2.0%, by weight, based on the weight of the composition. In some embodiments, ferulic acid is present in the composition in an amount that is at least about 0.5%, by weight, based on the weight of the composition.

In some embodiments, ascorbic acid is present in the composition in a range from 10% to about 20%, by weight, based on the weight of the composition. In some embodiments, ascorbic acid is present in the composition from at least about 10% to about 15%, by weight, based on the weight of the composition.

In some embodiments, salicylic acid is present in the composition in a range from about 0.1% to about 2.0%, by weight, based on the weight of the composition. In some embodiments, salicylic acid is present in the composition in a range from not more than about 0.40% to about 0.50%, by weight, based on the weight of the composition.

In some embodiments, the composition includes zinc PCA, present in the composition in a range from about 0.1% to about 2.0%, by weight, based on the weight of the composition. In some embodiments, zinc PCA is present in the composition in a range from not more than about 0.4% to about 0.8%, by weight, based on the weight of the composition.

In some embodiments, the composition includes solvent comprising water present in the composition in a range from about 20% to about 80%, by weight, based on the weight of the composition. In some embodiments, the composition includes solvent comprising water present in the composition in a range from about 40% to about 60%, by weight, based on the weight of the composition in some embodiments, the composition includes solvent comprising one or more of alcohol and glycol present in the composition in the range from about 5% to about 20%, by weight, based on the weight of the composition.

In some embodiments, the suitable carrier system is selected from oil-in-water emulsions, water-in-oil emulsions, silicone in water emulsions, dispersions, miceller waters, biphasic compositions, foams, and alcohol-based carriers. In some embodiments, composition is appropriate for topical application to the skin and is in the form of a lotion, serum, gel, milk, foam, liquid foundation or cream.

In some specific embodiments, the composition comprising an antioxidant acid system includes gallic acid, present in the composition is in an amount that is at least about 5%, ferulic acid, present in the composition in in an amount that is at least about 0.5%, ascorbic acid, present in the composition from at least about 10% to about 15%, and salicylic acid, present in the composition in a range from not more than about 0.40% to about 0.50%, all percentages by weight, based on the weight of the composition. In some specific embodiments, the compositions includes a suitable carrier system, is selected from oil-in-water emulsions, water-in-oil emulsions, silicone in water emulsions, dispersions, miceller waters, biphasic compositions, foams, and alcohol-based carriers.

In some specific embodiments, the compositions includes zinc PCA, present in the composition in a range from about 0.1% to about 2.0%, by weight, based on the weight of the composition.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### BRIEF DESCRIPTION OF THE DRAWINGS

Features and advantages of the general inventive concepts will become apparent from the following description made with reference to the accompanying drawings, including drawings represented herein in the attached set of FIGURES, of which the following is a brief description:

FIG. 1 is a bar graph showing comparative performance results demonstrating antioxidant activity.

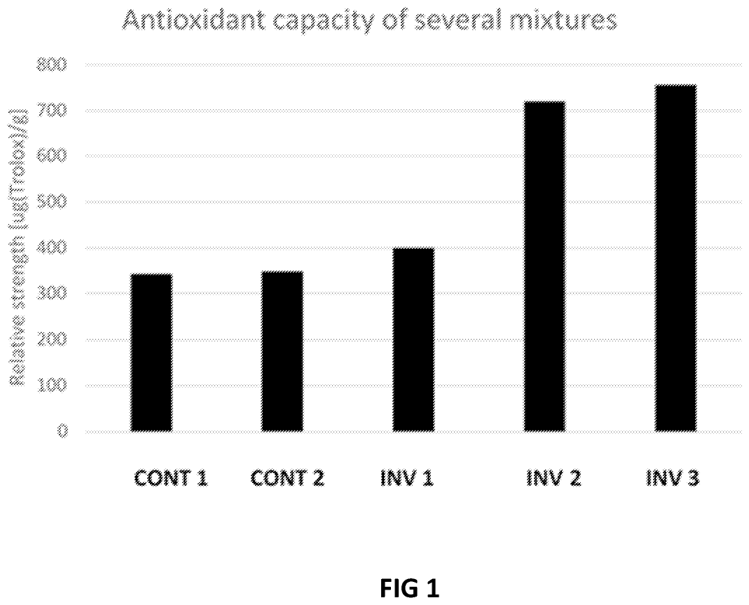

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

The disclosure provides a composition that includes a novel association of antioxidant acids demonstrates an unexpected synergistic antioxidant effect that is relevant for the topical treatment of signs of aging and reducing cutaneous lipid peroxidation. The composition includes (a) gallic acid, (b) ferulic acid, (c) ascorbic acid (Vitamin C), and (d) salicylic acid, all present in the composition in amounts sufficient to produce the synergistic antioxidant activity. In some embodiments, gallic acid is present in an amount that is in the range from about 1% to about 10%, and in some examples, in an amount that is at least about 5%. The composition may also contain additional antioxidants, additives and other components, as described herein below.

Applicants formulated a novel association of antioxidant acids that demonstrates an unexpected synergistic antioxidant effect that is relevant for the topical treatment of signs of aging and reducing cutaneous lipid peroxidation. The novel association may be particularly beneficial for treating oily and/or acne prone skin. The synergistic effect of the inventive association is plainly shown in comparative studies described herein in the Examples employing only sub combinations of the antioxidant acids, some of which sub combinations are known in the cosmetics art and which sub combinations do not evidence any predictable additive or synergistic benefit. The compositions provide stronger protective effects against free radicals and the damaging effects of reactive oxygen species in that the combinations in the compositions herein show synergistic antioxidant activity wherein the activity of an inventive composition comprising the full association of gallic acid, ferulic acid, ascorbic acid and salicylic acid is greater than the antioxidant activity of the combination including ferulic acid, ascorbic acid and salicylic acid, as shown in the Examples.

Since these tests were performed on water-based solutions, identified associations between the antioxidant acids may be applied in any cosmetic product to provide a stronger protection from free radicals. Cosmetic formulations comprising the novel antioxidant association that are appropriate for topical application to the skin include compositions in the form of a lotion, serum, gel, milk, foam, liquid foundation or cream.

Suitable cosmetic systems which include the novel disclosed antioxidant association include lotion or serum type of emulsions with a liquid or semi-liquid consistency of the milk type, obtained by dispersion of a fatty phase in an aqueous phase (O/W) or vice versa (W/O), including those employing silicone and non-silicone oils, or of suspensions or emulsions with a soft consistency of the aqueous gel or cream type, or of microcapsules or microparticles, or of vesicular dispersions of ionic and/or nonionic type or of foams, or they may be alcohol based, or they may be provided in the form of toners or miceller water, or they may be biphasic and include water and other solvents such as alcohols and other water-soluble solvents and oils that are not water miscible. Biphasic compositions require pre-application agitation to form an emulsion prior to application to the keratinous tissue, such as skin, which emulsion is of sufficient quality and stability to allow a uniform application of the emulsified phases. Thus, a suitable carrier system according to the disclosure may be selected from any one of oil-in-water emulsions, water-in-oil emulsions, silicone in water emulsions, dispersions, miceller waters, biphasic compositions, foams, and alcohol based.

Antioxidant Acids

Gallic Acid

In accordance with the various embodiments, the composition according to the disclosure comprises gallic acid.

Gallic acid (also known as 3,4,5-trihydroxybenzoic acid) is a trihydroxybenzoic acid, a type of phenolic acid, found in gallnuts, sumac, witch hazel, tea leaves, oak bark, and other plants. The chemical formula of gallic acid is C₆H₂(OH)₃COOH. It is found both free and as part of hydrolyzable tannins.

In accordance with the various embodiments, the amount of gallic acid present in the composition is in a range from about 1% to about 10%, or from about 2% to about 9%, or from about 3% to about 7%, or from about 4% to about 6%, or from about 4% to about 5%, or is about 5%, or any suitable combination, sub-combination, range, or sub-range thereof by weight, based on the weight of the composition. In accordance with some embodiments, the amount of gallic acid present is at least about 5% to about 10%.

Thus, any one of or a combination of gallic acid is present, by weight, based on the total weight of the composition, from about 1, 2, 3, 4, 5, 6, 7, 8, 9, to about 10 weight percent, including increments and ranges therein and there between.

Ferulic Acid

In accordance with the various embodiments, the composition according to the disclosure comprises ferulic acid.

Ferulic acid, which is a hydroxycinnamic acid that can be broadly found in giant fennel, the seeds of coffee, apple, artichoke, peanut, and oranges, as well as in both seeds and cell walls of commelinid plants (such as rice, wheat, oats, and pineapple). Like many natural phenols, it is a strong antioxidant that is very reactive toward free radicals and reduces oxidative stress.

In accordance with the various embodiments, the amount of ferulic acid present in the composition is in a range from about 0.1% to about 2.0%, or from about 0.2% to about 1.5%, or from about 0.3% to about 1.0%, or from about 0.35% to about 0.75%, or from about 0.4% to about 0.5%, or is about 0.5%, or any suitable combination, sub-combination, range, or sub-range thereof by weight, based on the weight of the composition. In accordance with some embodiments, the amount of ferulic acid present is at least about 0.5%.

Thus, any one of or a combination of ferulic acid is present, by weight, based on the total weight of the composition, from about 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9 to about 2.0 weight percent, including increments and ranges therein and there between.

Ascorbic Acid

In accordance with the various embodiments, the composition according to the disclosure comprises ascorbic acid.

Ascorbic Acid is a natural water-soluble vitamin (Vitamin C). Ascorbic acid is a potent reducing and antioxidant agent that functions in fighting bacterial infections, in detoxifying reactions, and in the formation of collagen in fibrous tissue, teeth, bones, connective tissue, skin, and capillaries.

In accordance with the various embodiments, the amount of ascorbic acid present in the composition is in a range from about 10% to about 20%, or from about 11% to about 19%, or from about 12% to about 18%, or from about 13% to about 17%, or from about 14% to about 16%, or is about 15%, or any suitable combination, sub-combination, range, or sub-range thereof by weight, based on the weight of the composition. In accordance with some embodiments, the amount of ascorbic acid present is at least about 10% to about 15%.

Thus, any one of or a combination of ascorbic acid is present, by weight, based on the total weight of the composition, from about 10, 11, 12, 13, 14, 15, 16, 17, 18, 19, to about 20 weight percent, including increments and ranges therein and there between.

Salicylic Acid

In accordance with the various embodiments, the composition according to the disclosure comprises salicylic acid.

Salicylic acid (from Latin salix, willow tree) is a lipophilic monohydroxybenzoic acid, a type of phenolic acid, and a beta hydroxy acid (BHA). It has the formula C₇H₆O₃. This colorless crystalline organic acid is widely used in organic synthesis and functions as a plant hormone.

In accordance with the various embodiments, the amount of salicylic acid present in the composition is in a range from about 0.1% to about 2.0%, or from about 0.2% to about 1.5%, or from about 0.3% to about 1.0%, or from about 0.35% to about 0.75%, or from about 0.4% to about 0.5%, or is about 0.5%, or any suitable combination, sub-combination, range, or sub-range thereof by weight, based on the weight of the composition. In accordance with some embodiments, the amount of salicylic acid present is not more than about 0.40% to about 0.50%.

Thus, any one of or a combination of salicylic acid is present, by weight, based on the total weight of the composition, from about 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9 to about 2.0 weight percent, including increments and ranges therein and there between.

Zinc PCA

In accordance with some various embodiments, the composition according to the disclosure may optionally include Zinc PCA. Zinc PCA is the Zinc salt of pyrrolidone carboxylic acid.

In accordance with the various embodiments, the amount of Zinc PCA, when present in the composition, is from about 0.1% to about 2.0%, or from about 0.2% to about 1.5%, or from about 0.3% to about 1.0%, or from about 0.4% to about 0.8%, or is about 0.5%, or any suitable combination, sub-combination, range, or sub-range thereof by weight, based on the weight of the composition. In accordance with some embodiments, the amount of Zinc PCA present is not more than about 0.4% to about 0.8%.

Thus, Zinc PCA, when present in the composition, is present, by weight, based on the total weight of the composition, from about 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9 to about 2.0 weight percent, including increments and ranges therein and there between.

Solvents

In accordance with the various embodiments, water is present in the composition in a range from about 20% to about 80%, or from about 25% to about 75%, or from about 30% to about 70%, or from about 35% to about 65%, or from about 40% to about 60%, or from about 45% to about 55%, or any suitable combination, sub-combination, range, or sub-range thereof by weight, based on the weight of the composition. One of ordinary skill in the art, however, will appreciate that other ranges are within the scope of the invention. Thus, water is present, by weight, based on the total weight of the composition, from about 20, 21, 22, 23, 24, 25, 26, 27, 28, 29, 31, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, to about 80 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the composition is not limited but is generally not less than 3.5. The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

In some embodiments, the composition includes one or more solvents comprising alcohol for solubilizing acid, for example, monoalcohols such as monohydric C₁-C₈ alcohols such as ethanol, propanol, butanol, isopropanol, isobutanol, and benzyl alcohol, and phenylethyl alcohol. In some embodiments, the alcohol solvent is selected from one or a combination of ethanol and isopropyl alcohol. The solvents may also include water-soluble solvents including polyols and short chain mono-alcohols. In some specific embodiments the water-soluble solvents are selected from glycerin, propylene glycol, butylene glycol, and combinations thereof. In one example, a solvent used in accordance with the disclosure is propylene glycol.

In accordance with the various embodiments, the amount of alcohol or glycol present in the composition is in the range from about 5% to about 20%, or from about 6% to about 18%, or from about 10% to about 15%, or any suitable combination, sub-combination, range, or sub-range thereof by weight, based on the weight of the composition. One of ordinary skill in the art, however, will appreciate that other ranges are within the scope of the invention. In some embodiments, denatured alcohol is present at about 15% and propylene glycol is present at about 7.5%.

Thus, any one of or a combination of alcohols or glycols may be present, by weight, based on the total weight of the composition, from about 5, 6, 7, 8, 9, 10, 11, 12, 13, 14, 15, 16, 17, 18, 19, to about 20 weight percent, including increments and ranges therein and there between.

Optional Additional Antioxidants and Additives

The compositions can also contain one or more additional antioxidants that is/are different from the antioxidant acids used in the composition. Additional antioxidants can be any antioxidant or other additive suitable for use in cosmetic formulations. Suitable additives include, but are not limited to, resveratrol, tannic acid, polyphenols, amino acids and derivatives thereof, imidazoles, peptides such as carnosine and derivatives, carotenoids, carotenes (such as α-carotene, β-carotene, and lycopene), additional acids selected from α-hydroxy acids (such as citric acid, lactic acid, malic acid, and combinations of these), beta-hydroxy acids (such as propionic acid, beta-hydroybutyric acid, beta-hydroxy beata-methylbutyric acid, carnitine, and combinations of these), tocopherols and derivatives (such as Vitamin E), vitamin A, co-enzyme Q10, bioflavonoids, glutathione, plant extracts (such as rosemary extract, olive leaf extracts, Scutellaria Baicalensis (baicalin), and Mango Leaf Extract (mangiferin), and green tea extracts.

The number of additional antioxidants present in the compositions can range from about 0.01% to about 20%, or from about 0.1% to about 20%, or from about 0.01% to about 10%, based on the total weight of the composition. In some embodiments, the compositions do not comprise any additional antioxidants.

Optional Additives

The compositions can also comprise at least one additive conventionally used in the cosmetics field which does not affect the properties of the compositions according to the invention, such as thickeners, pearlescent agents, preservatives, sunscreens, anionic or nonionic or cationic or amphoteric polymers, proteins, protein hydrolysates, fatty acids, silicones, vegetable, mineral or synthetic oils, gelling agents, fragrances, fillers, screening agents, odor absorbers and coloring materials.

Any one or a combination of additives can be present in the composition according to the invention in proportions which are not limited, but which advantageously fall in the range from 0% to 50% by weight, or 1 to 50% by weight, with respect to the total weight of the composition.

Formulations Comprising the Compositions

Generally, any composition of the invention can be ingested, injected or topically applied to the skin (over any cutaneous region of the body) or to the mucous membranes (oral, jugal, gingival, genital, conjunctival, and the like). Depending on the method of administration under consideration, the composition can be provided in any dosage form normally used.

For topical application to the skin, the composition can have the form in particular of aqueous or oily solutions or of dispersions of the lotion or serum type of emulsions with a liquid or semi-liquid consistency of the milk type, obtained by dispersion of a fatty phase in an aqueous phase (O/W) or vice versa (W/O), including those employing silicone and non-silicone oils, or of suspensions or emulsions with a soft consistency of the aqueous gel or cream type, or of microcapsules or microparticles, or of vesicular dispersions of ionic and/or nonionic type or of foams, or they may be alcohol based, or they may be provided in the form of toners or miceller water, or they may be biphasic and include water and other solvents such as alcohols and other water-soluble solvents and oils that are not water miscible. Biphasic compositions require pre-application agitation to form an emulsion prior to application to the keratinous tissue, such as skin, which emulsion is of sufficient quality and stability to allow a uniform application of the emulsified phases. Thus, a suitable carrier system according to the disclosure may be selected from any one of oil-in-water emulsions, water-in-oil emulsions, silicone in water emulsions, dispersions, miceller waters, biphasic compositions, foams, and alcohol based. These compositions are prepared according to the usual methods.

For injection, the composition can be provided in the form of aqueous or oily lotions or in the form of serums. For the eyes, the composition can be provided in the form of drops and, for ingestion, it can be provided in the form of capsules, granules, syrups or tablets.

The amounts of the various constituents of the compositions according to the invention are those conventionally used in the fields under consideration.

In the cosmetics field, these compositions constitute in particular creams for cleaning, protecting, treating or caring for the face, for the hands, for the feet, for the major anatomical folds or for the body (for example, day creams, night creams, make-up-removing creams, foundation creams or sun creams), liquid foundations, make-up-removing milks, protective or care body milks, sun milks, lotions, gels or foams for caring for the skin, such as cleansing lotions, sun lotions, artificial tanning lotions, bath compositions, deodorizing compositions comprising a bactericidal agent, aftershave gels or lotions, depilatory creams, compositions for countering insect stings or bites, pain-relieving compositions or compositions for treating one or more of aging, sun damaged, oily and acne prone skin, and certain skin diseases, such as eczema, rosacea, psoriasis, lichen and severe pruritus.

### EXAMPLES

### Example 1: Inventive and Control Compositions

TABLE 1
Inventive Compositions

| | INV 1 | INV 2 | INV 3 |
|---|---|---|---|
| | 1% Gallic | 5% Gallic | 5% Gallic |
| | Acid, with | Acid, with | Acid, with |
| INCI | no Zinc PCA | Zinc PCA | no Zinc PCA |
| ASCORBIC ACID | 15.0 | 15.0 | 15.0 |
| WATER/AQUA | 60.5 | 56.0 | 56.5 |
| ALCOHOL DENAT. | 15.0 | 15.0 | 15.0 |
| SALICYLIC ACID | 0.5 | 0.5 | 0.5 |
| PROPYLENE | 7.5 | 7.5 | 7.5 |
| GLYCOL | | | |
| ZINC PCA | 0.0 | 0.5 | 0.0 |
| FERULIC ACID | 0.5 | 0.5 | 0.5 |
| GALLIC ACID | 1.0 | 5.0 | 5.0 |

TABLE 1
Control Compositions

| | CONTROL 1 | |
|---|---|---|
| | No Gallic Acid, | CONTROL 2 |
| INCI | No Zinc PCA | No Gallic Acid |
| ASCORBIC ACID | 15.0 | 15.0 |
| WATER/AQUA | 61.5 | 61.0 |
| ALCOHOL DENAT. | 15.0 | 15.0 |
| SALICYLIC ACID | 0.5 | 0.5 |
| PROPYLENE GLYCOL | 7.5 | 7.5 |
| ZINC PCA | 0.0 | 0.5 |
| FERULIC ACID | 0.5 | 0.5 |
| GALLIC ACID | 0 | 0 |

### Example 2: Measurement of Antioxidant Capacity of Inventive and Control Compositions

As shown in Table 3, the AOX components of the compositions are listed along with the results of the antioxidant strength testing employing an oxygen radical absorbance capacity (ORAC) assay which allows for the quantification of the capacity of an antioxidant to quench the peroxyl radical. The peroxyl radical, generated from the thermal decomposition of 2,2′-azobis-2-methyl-propanimidamide dihydrochloride (AAPH), will quench a fluorescent probe by a hydrogen atom transfer reaction. In the presence of an antioxidant, however, the molecule will initially block or prevent the quenching of the probe, causing a delay in the fluorescence decay profile. The area under the fluorescence decay curves for samples with and without the presence of an antioxidant are compared to that of a standard reference material and the peroxyl radical antioxidant capacity is determined. References disclosing ORAC assays include Cao G, Alessio H, Cutler R (1993). “Oxygen-radical absorbance capacity assay for antioxidants”. Free Radic Biol Med 14 (3): 303-11; Ou B, Hampsch-Woodill M, Prior R (2001). “Development and validation of an improved oxygen radical absorbance capacity assay using fluorescein as the fluorescent probe”. J Agric Food Chem 49 (10): 4619-26; Huang D, Ou B, Prior R (2005). “The chemistry behind antioxidant capacity assays”. J. Agric. Food Chem. 53 (6): 1841-56; and Garrett A R, Murray B K, Robison R A, O'Neill K L (2010). “Measuring antioxidant capacity using the ORAC and TOSC assays”. Advanced Protocols in Oxidative Stress II: Methods in Molecular Biology (series), Donald J Armstrong (ed) 594: 251-62.

TABLE 3
Results of AOX Strength Testing

| | | | | | | Relative |
|---|---|---|---|---|---|---|
| Test | Gallic | Ferulic | Ascorbic | Salicylic | Zinc | AOX |
| Comp | Acid | Acid | Acid | Acid | PCA | Strength |
| Control | 0% | 0.5% | 15% | 0.5% | 0% | 340 |
| 1 | | | | | | |
| Control | 0% | 0.5% | 15% | 0.5% | 0.5% | 345 |
| 2 | | | | | | |
| Inventive | 1% | 0.5% | 15% | 0.5% | 0% | 400 |
| 1 | | | | | | |
| Inventive | 5% | 0.5% | 15% | 0.5% | 0.5% | 720 |
| 2 | | | | | | |
| Inventive | 5% | 0.5% | 15% | 0.5% | 0% | 750 |
| 3 | | | | | | |

Ascorbic acid is well known to have excellent antioxidant strength. The addition of 5% gallic acid to an already very high concentration of ascorbic acid (15%) resulted in a substantial increase in the overall antioxidant capacity of the association. The effect was modest when 1% gallic acid was employed, but significant when 5% gallic acid was employed. This association and concentration is unique in its observed performance. Zinc PCA was not found to be a contributing factor for antioxidant potential.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“One or more,” as used herein, means at least one, and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “from about 1% to about 10%, or from about 2% to about 8%, or from about 3% to about 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “from about 1% to about 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a certain embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. A composition comprising an antioxidant acid system, the composition comprising:
(a) an antioxidant acid system comprising:
i. gallic acid,
ii. ferulic acid,
iii. ascorbic acid, and
iv. salicylic acid, and
(b) a suitable carrier system,
wherein the antioxidant acid system components are present in the composition in amounts sufficient to produce synergistic antioxidant activity.

2. The composition according to claim 1, wherein gallic acid is present in the composition in a range from about 1% to about 10%, by weight, based on the weight of the composition.

3. The composition according to claim 1, wherein the amount of gallic acid present in the composition is in an amount that is at least about 5% to about 10%, by weight, based on the weight of the composition.

4. The composition according to claim 1, wherein the amount of gallic acid present in the composition is in an amount that is at least about 5%, by weight, based on the weight of the composition.

5. The composition according to claim 1, wherein ferulic acid is present in the composition in a range from about 0.1% to about 2.0%, by weight, based on the weight of the composition.

6. The composition according to claim 1, wherein ferulic acid is present in the composition in an amount that is at least about 0.5%, by weight, based on the weight of the composition.

7. The composition according to claim 1, wherein ascorbic acid is present in the composition in a range from 10% to about 20%, by weight, based on the weight of the composition.

8. The composition according to claim 1, wherein ascorbic acid is present in the composition from at least about 10% to about 15%, by weight, based on the weight of the composition.

9. The composition according to claim 1, wherein salicylic acid is present in the composition in a range from about 0.1% to about 2.0%, by weight, based on the weight of the composition.

10. The composition according to claim 1, wherein salicylic acid is present in the composition in a range from not more than about 0.40% to about 0.50%, by weight, based on the weight of the composition.

11. The composition according to claim 1, further comprising zinc PCA, present in the composition in a range from about 0.1% to about 2.0%, by weight, based on the weight of the composition.

12. The composition according to claim 1, further comprising zinc PCA, present in the composition in a range from not more than about 0.4% to about 0.8%, by weight, based on the weight of the composition.

13. The composition according to claim 1, wherein solvent comprising water is present in the composition in a range from about 20% to about 80%, by weight, based on the weight of the composition.

14. The composition according to claim 1, wherein solvent comprising water is present in the composition in a range from about 40% to about 60%, by weight, based on the weight of the composition.

15. The composition according to claim 1, wherein solvent comprising one or more of alcohol and glycol is present in the composition in the range from about 5% to about 20%, by weight, based on the weight of the composition.

16. The composition according to claim 1, wherein the suitable carrier system is selected from oil-in-water emulsions, water-in-oil emulsions, silicone in water emulsions, dispersions, miceller waters, biphasic compositions, foams, and alcohol-based carriers.

17. The composition according to claim 16, wherein the composition is appropriate for topical application to the skin and is in the form of a lotion, serum, gel, milk, foam, liquid foundation or cream.

18. A composition comprising an antioxidant acid system, the composition comprising:
(a) an antioxidant acid system comprising:
i. gallic acid, present in the composition is in an amount that is at least about 5%,
ii. ferulic acid, present in the composition in in an amount that is at least about 0.5%,
iii. ascorbic acid, present in the composition from at least about 10% to about 15%, and
iv. salicylic acid, present in the composition in a range from not more than about 0.40% to about 0.50%,
all percentages by weight, based on the weight of the composition, and
(b) a suitable carrier system,
wherein the antioxidant acid system components are present in the composition in amounts sufficient to produce synergistic antioxidant activity.

19. The composition according to claim 18, further comprising zinc PCA, present in the composition in a range from about 0.1% to about 2.0%, by weight, based on the weight of the composition.

20. The composition according to claim 19, wherein the suitable carrier system is selected from oil-in-water emulsions, water-in-oil emulsions, silicone in water emulsions, dispersions, miceller waters, biphasic compositions, foams, and alcohol-based carriers.
